(19) United States
(12) Patent Application Publication
(10) Pub. No.: US 2021/0008124 A1
(43) Pub. Date: Jan. 14, 2021

(54) COMPOSITION FOR RELIEVING STRESS AND ENHANCING IMMUNITY AND PREPARATION METHOD THEREOF

(71) Applicant: Yantai Institute of Coastal Zone Research, Chinese Academy of Sciences, Yantai (CN)

(72) Inventors: JIAN PENG, Yantai (CN); SUYUN FANG, Yantai (CN); XIAOKE HU, Yantai (CN); JUN MIN, Yantai (CN); HAIKUN ZHANG, Yantai (CN); CAIXIA WANG, Yantai (CN); YAN LI, Yantai (CN); WEI LIU, Yantai (CN)

(21) Appl. No.: 16/897,378

(22) Filed: Jun. 10, 2020

(30) Foreign Application Priority Data
Jul. 11, 2019 (CN) 201910625434.3

(51) Int. Cl. A61K 35/616; A61K 36/9066; A61K 36/05; A61K 36/45; A61K 36/258; A61K 36/889; A61K 31/405; A61K 47/24; A61P 37/04

(52) U.S. Cl. CPC A61K 35/616; A61K 36/9066; A61K 36/05; A61K 36/45; A61K 36/258; A61K 2236/53; A61K 31/405; A61K 47/24; A61P 37/04; A61K 2236/37; A61K 2236/333; A61K 36/889

(57) ABSTRACT

The invention belongs to the technical field of health food and particularly relates to a composition for relieving stress and enhancing immunity and a preparation method thereof. The composition for relieving stress and enhancing immunity is calculated in parts by weight, 100-300 parts of a radix curcuma extract, 100-250 parts of a sea cucumber extract, 100-250 parts of a chlorella extract, 50-150 parts of a cranberry extract, 100-300 parts of a ginseng extract, 80-150 parts of a coconut oil extract and 100-200 parts of a palm oil extract. The composition can significantly enhance the body's immunity, relieve mental stress and improve the level of human health. In addition, the composition of the present invention is suitable for long-term use and has a small adverse reaction.

### CROSS-REFERENCE TO RELATED PATENT APPLICATION

This application claims the benefit of priority to China Patent Application No. 201910625434.3, filed on Jul. 11, 2019 in People's Republic of China. The entire content of the above identified application is incorporated herein by reference.

### TECHNICAL FIELD

The invention belongs to the technical field of health foods, and particularly relates to a healthcare product for relieving stress and enhancing immunity and a preparation method thereof.

### BACKGROUND

With the development of society, people's living standards are constantly improving, but the problems of life that come with it are becoming more and more obvious. The high-speed life rhythm and economic growth not only bring us opportunities, but also bring some effects on our psychology. While enjoying the prosperity of material life, people have also suffered more psychological pressure than ever before. The ubiquitous competition, the lack of emotional communication, the frustration of business and love, the variability of the living environment, the poor social interaction, and the imbalance of economic income all may cause modern people to fall into anxiety and pessimism. At present, nearly 70% of people in China are in a sub-health state, especially those with high work and life pressures and low immunity, whose health is greatly threatened by potential.

Long-term tension and high pressure will cause the human body to be in a state of chronic stress, leading to persistent psychological tension, insomnia and overwork, further leading to imbalance in diet, thereby disturbing the rhythm of life, gradually deteriorating physical and mental state, and making the body immune surveillance function reduced, immunity reduced, and ultimately cause human disease. Modern medical research has fully proved that low immunity is the source of all diseases and improving immunity to prevent and cure various diseases is the most fundamental way of health. Therefore, it is especially important to relieve stress and improve self-immunity in a timely manner, traditional methods such as chatting with friends, going out for a picnic, and exercising are good choices. To a certain extent, the inhalation of functional substances to relieve stress and enhance human immunity has become one of the indispensable ways for the contemporary social population. Therefore, it is essential to develop a functional substance that can meet people's needs to relieve stress and enhance immunity with high safe performance, overall conditioning, and low-cost.

### SUMMARY

In view of the above, the present invention provides a composition for relieving stress and enhancing immunity and a preparation method thereof.

A first object of the present invention is to provide a composition for relieving stress and enhancing immunity;

A second object is to provide a method for preparing the composition for relieving stress and enhancing immunity;

A third object is to provide a formulation of the composition for relieving stress and enhancing immunity;

A fourth object is to provide a use of the composition for relieving stress and enhancing immunity.

To achieve the above objects, the present invention provides the following technical solutions:

A composition for relieving stress and enhancing immunity, the composition for relieving stress and enhancing immunity is calculated in parts by weight, 100-300 parts of a turmeric extract, 100-250 parts of a sea cucumber extract, 100-250 parts of a chlorella extract, 50-150 parts of a cranberry extract, 100-300 parts of a ginseng extract, 80-150 parts of a coconut oil extract, and 100-200 parts of a palm oil extract.

The composition for relieving stress and enhancing immunity further comprises, in parts by weight, 50-100 parts of lecithin and 50-80 parts of 5-hydroxytryptophan.

The composition for relieving stress and enhancing immunity is calculated in parts by weight, 150-250 parts of a turmeric extract, 100-200 parts of a sea cucumber extract, 150-200 parts of a chlorella extract, and 80-100 parts of a bilberry extract, 150-250 parts of a ginseng extract, 100-150 parts of a coconut oil extract, 150-200 parts of a palm oil extract, 50-100 parts of lecithin, and 50-80 parts of 5-hydroxytryptophan.

Mixing the turmeric extract, sea cucumber extract, chlorella extract, cranberry extract, ginseng extract, coconut oil extract, and palm oil extract in proportion according to the above ratio, then adding the formula-proportioned lecithin and 5-hydroxytryptophan and mixing uniformly to obtain a target composition for relieving stress and enhancing immunity.

The turmeric extract is prepared by pulverizing a turmeric medicinal material, extracting the turmeric medicinal material with a mixed solution of 50%-95% lower alcohol solution and n-hexane, the extraction time is 1-3 hours, the extraction temperature is 50° C.-85° C., and the extraction times are 2-3 times; collecting a concentrated extract, and adding a 80%-90% lower alcohol solution having a volume of 0.2-0.4 times of the concentrate, stirring uniformly and allowing to stand for 12-24 hours, centrifuging, and collecting crystals; washing the crystals again with a 80%-95% lower alcohol solution, and centrifuging to obtain crystals; drying the crystal at a low temperature to obtain a turmeric extract;

The sea cucumber extract is prepared by washing a sea cucumber with cold water, adding cold purified water at 4° C. to the sea cucumber and sealing, and soaking in cold storage at 4° C. for 48 hours; then homogenizing, and freezing at −20° C. until completely frozen, then thawing at room temperature, centrifuging at 4° C. and collecting a supernatant and a precipitate; filtering the supernatant through a sterile membrane with a pore size of 0.25 μm, and storing a filtrate at 4° C. for use; freezing and thawing the resulting precipitate again by adding water, centrifuging and collecting a supernatant; blending the two supernatants and concentrating under reduced pressure, filtering and lyophilizing a concentrated sea cucumber extract to obtain a sea cucumber extract;

The chlorella extract is prepared by pulverizing a chlorella, dispersing uniformly with water having a weight of 10-12 times of the chlorella to obtain a dispersion; centrifuging and separating the dispersion, taking a supernatant, filtering, decolorizing and lyophilizing to obtain a chlorella extract.

The cranberry extract is prepared by pulverizing a cranberry, extracting with an acidic methanol, extracting at 80-100° C. for 10-20 minutes, and then drying into a powder;

The ginseng extract is prepared by pulverizing a ginseng, soaking in water, vacuum distilling, stopping distillation when a volume of a distillate is half of the amount of added water, and collecting the distillate; and then continuously adding water to a liquid, vacuum distilling, stopping distillation when a volume of a distillate is half of the amount of added water, and collecting the distillate; blending the two distillates to obtain a ginseng extract.

The coconut oil extract is prepared by repeatedly extracting a natural coconut oil by alcohol, and lyophilizing an extract to obtain a coconut oil extract;

The palm oil extract is prepared by repeatedly extracting a natural palm oil by alcohol and lyophilizing an extract to obtain a palm oil extract.

The composition for relieving stress and enhancing immunity is used for the application of preparing a drug, food and health food for relieving body stress and enhancing immunity.

A formulation for relieving stress and enhancing immunity, the composition is prepared as an active ingredient together with pharmaceutically acceptable adjuvants to obtain a forming agent; wherein, a content of the active ingredient is from 16 wt %-40 wt %.

A dosage form is a tablet, a capsule, a granule, a pill or a powder.

The composition for relieving stress and enhancing immunity is used for the application of preparing a drug, food and health food for relieving body stress and enhancing immunity.

The turmeric of the composition of the present invention is a ginger plant. There are three natural phytochemicals of curcumin, demethoxycurcumin and bis-demethoxycurcumin in the turmeric, collectively referred to as curcuminoid. Modern medical research shows that in addition to dispelling the effects of alcohol and preventing hangover, turmeric has a wide range of pharmacological effects, such as anti-inflammatory, anti-pathogenic microorganisms, anti-oxidation, lipid-lowering, liver-protecting, gallbladder-nourishing, and cardiovascular and cerebrovascular-protecting. Turmeric is a commonly used traditional Chinese medicine, it is recorded in Chinese Materia Medica: “bitter and acrid in flavor; warm in nature, acts on spleen and liver. It has the efficacies of invigorating blood and circulating Qi.” Chinese Pharmacopoeia records: “for pain in chest and hypochondrium, amenorrhea, phlegm, rheumatism, pain in shoulder and arm, and tumble swelling.”

Sea cucumber is a marine echinoderm living in the sea to 8000 meters. It has a history of more than 600 million years and is one of the world's eight treasures (shark fin, abalone, fish lip, skirt, scallop, fish crisp, Chinese wood-frog, and sea cucumber), and is a valuable traditional Chinese medicine, which has the functions of improving memory, delaying gonad senescence, preventing arteriosclerosis and anti-tumor. According to the Supplement to Compendium of Materia Medica records: sea cucumber, sweet and salty in flavor, tonifying kidney, strengthening and nourishing marrow and essence, helping urination, aphrodisiac treatment, warm in nature, and enough to act as ginseng. From the perspective of Chinese medicine, sea cucumber has the effects of nourishing Yin, tonifying kidney, strengthening yang, nourishing essence, replenishing blood and moistening dryness. It has good health effects for those with physical weakness.

Chlorella, commonly known as green algae, is one of the major algae species in the strategic emerging industries of microalgae energy and microalgae carbon fixation that are developing and cultivating at home and abroad. Chlorella growth factor has a series of effects such as promoting cell growth and patient recovery, enhancing immune function, detoxification, lowering blood-sugar and blood-pressure.

Cranberry is wild bilberry, azalea genus Vaccinium, and there are many sub-species of cranberry around the world, including: bilberry, American cranberry, Siberian cranberry, oval cranberry and blueberry, more than 400 varieties, especially in Sweden, Finland, Norway, Austria, and Switzerland, are preferred. Wild bilberry contains a large number of flavonoids, of which anthocyanins are the main type. Anthocyanins can reduce eye fatigue, improve night vision, and prevent cataracts. In addition, anthocyanins also have a strong ability to scavenge reactive oxygen radicals, strengthen capillaries, improve blood circulation, prevent thrombosis, reduce the frequency of arteriosclerosis, and fight cancer.

Ginseng is a ginseng of the Araliaceae plant. It is sweet and slightly bitter in flavor, acts on spleen, lung and heart, the ginseng has the functions of invigorating Qi, recovering the pulse condition and strengthening the vitality, tonifying the spleen and benefiting the lung, promoting the production of the body fluid and tranquilizing the mind, and is a traditional Chinese medicine for tonic. It has an application history of more than 4,000 years, and all the herbal documents of ancient China have been recorded. The Shenlong Materia Medica lists ginseng as the top grade; the Food Materia Medica records: “Ginseng is sweet in flavor and slightly cold in nature, non-toxic. Mainly benefit heart, tranquillize the mind, calm the soul, stop the horror, remove the evil spirits, improve eyesight, happiness and intelligence, light body and extend years after long-term use.” Ginseng can tonify Qi, invigorating the spleen and stomach, enhance body's disease resistance, and therefore have the function of enhancing immunity.

Coconut oil is virgin coconut oil with coconut flavor. The main ingredient is lauric acid, it is only found in breast milk and a few foods in nature. It can improve immunity, is beneficial to the human body and is harmless, and is one of healthy fats. Coconut oil is a beauty product for weight loss, it can be used for beauty and anti-aging. It has strong antioxidant capacity and can help the body to prevent the production of free radicals. It can lose weight in a healthy way, and it is easy to be used as energy combustion conversion without lipase decomposed. It can both moisturize the skin, adjust the secretion of sebaceous glands and improve the dry skin by internal or external use. It is a moisturizing hair care product and a pure massage oil. It does not increase the body burden and is highly exclusive to toxins. Therefore, it is effective to gradually eliminate toxins accumulated in the intestines, stomach and body by consuming pure coconut oil, and is the most natural way to maintain health.

Palm oil is extracted from palm fruits on oil palm trees. The oil squeezed from the pulp is called palm oil, the oil squeezed from the nuts is called palm kernel oil, and the compositions of the two oils are very different. Palm oil is also known as saturated fat because it contains 50% saturated fat. The fat is composed of a mixture of saturated fat, monounsaturated fat and polyunsaturated fat. The body's digestion and absorption rate of palm oil is over 97%. Palm oil itself does not contain cholesterol and is rich in antioxidants such as vitamins A and E. It protects against free radicals and helps protect skin cells and promote overall health.

The biological name of lecithin is phosphatidylcholine, which is composed of a fatty acid group, a glyceryl group, a phosphate group and a choline. The lecithin is rich in unsaturated fatty acids, acts as a free radical scavenging agent, and is capable of repairing the damaged biofilm system. As a precursor of acetylcholine, phosphatidylcholine can effectively increase the choline content in the blood and brain and promote the synthesis of acetylcholine from in vitro intake, thus playing the role of benefiting brain and intelligence. Lecithin is of great significance in inhibiting the inflammation, tumors, protection of damaged liver and improving the immune function, as well as the prevention of cardiovascular and cerebrovascular diseases.

The chemical name of 5-hydroxytryptophan is 5-hydroxy-3-indolyl-α-aminopropionic acid, a natural amino acid extracted from the dried seeds of the African mesquite plant Ghana grain. 5-hydroxytryptamine is a neurotransmitter responsible for the transmission of signals between cells. The level of 5-hydroxytryptamine in the human brain is closely related to human health. Studies have shown that low levels of 5-hydroxytryptamine can cause a variety of diseases. 5-hydroxytryptophan can help the body to increase the level of 5-hydroxytryptamine, can help relieve stress, improve the symptoms of fibroids, help to lose weight, lower blood pressure, prevent headaches and relieve symptoms of insomnia.

In summary, the advantages of the present invention are:

The composition of the present invention contains turmeric extract, sea cucumber extract, chlorella extract, cranberry extract, ginseng extract, coconut oil extract, lecithin and 5-hydroxytryptophan; The effects of materials on relieving stress and enhancing immunity are not only the classical medical records but also the clinical and modern pharmacological research foundations. The compound materials in the compound compatibility complement each other in efficacy, and have the functions of protecting liver and nourishing gallbladder, benefiting essence and nourishing Yin, tonifying spleen and stomach, nourishing the skin and detoxifying, through the role of strengthening the body resistance, invigorating Qi and strengthening the vitality. The experimental results show that the composition can significantly enhance the body's immunity, relieve mental stress and improve the level of human health. Further, the composition of the present invention is suitable for long-term use and has a small adverse reaction.

### DESCRIPTION OF THE EMBODIMENTS

The technical solutions of the present invention are further described below in conjunction with the embodiments. The described embodiments are only a part of the embodiments of the present invention, but the scope of protection of the present invention is not limited thereto, and any changes or substitutions based on the present invention belong to the scope of protection of the present invention.

The composition for relieving stress and enhancing immunity according to the present invention comprises in parts by weight, 100-300 parts of a turmeric extract, 100-250 parts of a sea cucumber extract, 100-250 parts of a chlorella extract, 50-150 parts of a cranberry extract, 100-300 parts of a ginseng extract, 80-150 parts of a coconut oil extract, and 100-200 parts of a palm oil extract.

The raw material further comprises 50-100 parts by weight of lecithin and 50-80 parts by weight of 5-hydroxytryptophan.

The composition for relieving stress and enhancing immunity comprises in parts by weight, 150-250 parts of a turmeric extract, 100-200 parts of a sea cucumber extract, 150-200 parts of a chlorella extract, 80-100 parts of a bilberry extract, 150-250 parts of a ginseng extract, 100-150 parts of a coconut oil extract, 150-200 parts of a palm oil extract, 50-100 parts of lecithin, and 50-80 parts of 5-hydroxytryptophan.

The method for preparing a composition for relieving stress and enhancing immunity according to the present invention, specifically includes:

The preparation of the turmeric extract comprises the steps of: pulverizing a turmeric medicinal material, extracting the turmeric medicinal material with a mixed solution of 50%-95% lower alcohol solution and n-hexane, the extraction time is 1-3 hours, the extraction temperature is 50° C.-85° C., and the extraction times are 2-3 times; collecting a concentrated extract, and adding a 80%-90% lower alcohol solution having a volume of 0.2-0.4 times of the concentrate, stirring uniformly and allowing to stand for 12-24 hours, centrifuging, and collecting crystals; washing the crystals again with a 80%-95% lower alcohol solution, and centrifuging to obtain crystals; drying the crystals at a low temperature, pulverizing, and sieving to obtain a turmeric extract;

The preparation of the sea cucumber extract comprises the steps of: washing a sea cucumber with cold water, adding cold purified water to the sea cucumber and sealing, a solid-liquid ratio of 1:5, and soaking in cold storage at 4° C. for 48 hours; then homogenizing the soaked sea cucumber using a homogenizer, and freezing at −20° C. until completely frozen, then thawing at room temperature, centrifuging at 4° C. and collecting a supernatant and a precipitate; filtering the supernatant through a sterile membrane with a pore size of 0.25 μm, and storing a filtrate at 4° C. for use; freezing and thawing the resulting precipitate again by adding water, centrifuging and collecting a supernatant; blending the two supernatants and concentrating under reduced pressure, and filtering the concentrated sea cucumber extract through a sterile membrane with a pore size of 0.25 μm and lyophilizing the filtrate in a lyophilizer to a water content of less than 3% to obtain a sea cucumber extract;

The preparation of the chlorella extract comprises the steps of: pulverizing a chlorella, dispersing uniformly with water having a weight of 10-12 times of the chlorella, sterilizing at 121° C. for 30 minutes, and cooling to room temperature to obtain a dispersion; centrifuging and separating the dispersion, taking a supernatant and filtering with diatomaceous earth; placing in a cold storage at 4° C. for 48 h, filtering again with diatomaceous earth, decolorizing with 200 mesh activated carbon for 30 min, filtering through a 0.22 μm filterable membrane, lyophilizing the filtrate in a lyophilizer to a water content of less than 3% to obtain a chlorella extract.

The preparation of the cranberry extract comprises the steps of: pulverizing the cranberry, extracting with acidic methanol, extracting at 80-100° C. for 10-20 minutes, and then drying into a powder;

The preparation of the ginseng extract comprises the steps of: dicing a ginseng, refluxing and extracting 3 times with 75% ethanol having a volume of 4 times, blending the extracts, concentrating the ethanol to a syrupy thick liquid, adding ethanol with a volume percentage concentration of 95%, making the alcohol content reach to 80% (in weight percent), placing for 24 h, filtering and concentrating to a syrupy thick paste to obtain a ginseng extract.

The preparation of the coconut oil extract comprises the following steps of: adding 80% methanol to a natural coconut oil, extracting for 20 min, centrifuging at 5000 r/min for 30 min, collecting a supernatant, repeating the extraction 3 times, and blending the supernatants, removing the methanol solvent by rotary evaporation at 35° C. and lyophilizing to obtain a coconut oil extract.

The preparation of the palm oil extract comprises the following steps of: adding 80% methanol to a natural palm oil, extracting for 20 min, centrifuging at 5000 r/min for 30 min, collecting a supernatant, repeating the extraction 3 times, and blending the supernatants, removing the methanol solvent by rotary evaporation at 35° C. and lyophilized to give a palm oil extract.

The formulation of the composition for relieving stress and enhancing immunity according to the present invention is prepared by adding pharmaceutically acceptable adjuvants to the composition for relieving stress and enhancing immunity to prepare tablets, capsules, granules, pills and powder.

The use of the composition for relieving stress and enhancing immunity according to the present invention is the use of the composition for relieving stress and enhancing immunity in preparing a drug, food and health food for relieving human body stress and enhancing immunity.

The present invention is further described below with specific implementation embodiments:

### Embodiment 1

100 g turmeric extract, 100 g sea cucumber extract, 100 g chlorella extract, 50 g cranberry extract, 100 g ginseng extract, 80 g coconut oil extract, and 100 g palm oil extract were weighed, mixed the components uniformly, then, added 50 g lecithin and 50 g 5-hydroxytryptophan, and mixed uniformly to obtain a target composition for relieving stress and enhancing immunity.

### Embodiment 2

300 g turmeric extract, 250 g sea cucumber extract, 250 g chlorella extract, 150 g cranberry extract, 300 g ginseng extract, 150 g coconut oil extract, and 200 g palm oil extract were weighed, mixed the components uniformly, then, added 100 g lecithin and 80 g 5-hydroxytryptophan, and mixed uniformly to obtain a target composition for relieving stress and enhancing immunity.

### Embodiment 3

200 g turmeric extract, 175 g sea cucumber extract, 175 g chlorella extract, 100 g cranberry extract, 200 g ginseng extract, 115 g coconut oil extract, and 150 g palm oil extract were weighed, mixed the components uniformly, then, added 75 g lecithin and 65 g 5-hydroxytryptophan, and mixed uniformly to obtain a target composition for relieving stress and enhancing immunity.

### Embodiment 4

100 g turmeric extract, 250 g sea cucumber extract, 175 g chlorella extract, 100 g cranberry extract, 200 g ginseng extract, 150 g coconut oil extract, and 100 g palm oil extract were weighed, mixed the components uniformly, then, added 100 g lecithin and 80 g 5-hydroxytryptophan, and mixed uniformly to obtain a target composition for relieving stress and enhancing immunity.

### Embodiment 5

300 g turmeric extract, 100 g sea cucumber extract, 175 g chlorella extract, 50 g cranberry extract, 100 g ginseng extract, 115 g coconut oil extract, and 150 g palm oil extract were weighed, mixed the components uniformly, then, added 75 g lecithin and 50 g 5-hydroxytryptophan, and mixed uniformly to obtain a target composition for relieving stress and enhancing immunity.

### Embodiment 6

200 g turmeric extract, 100 g sea cucumber extract, 250 g chlorella extract, 150 g cranberry extract, 300 g ginseng extract, 80 g coconut oil extract, and 200 g palm oil extract were weighed, mixed the components uniformly, then, added 50 g lecithin and 50 g 5-hydroxytryptophan, and mixed uniformly to obtain a target composition for relieving stress and enhancing immunity.

### Embodiment 7

100 g turmeric extract, 250 g sea cucumber extract, 175 g chlorella extract, 50 g cranberry extract, 300 g ginseng extract, 115 g coconut oil extract, and 100 g palm oil extract were weighed, mixed the components uniformly, then, added 100 g lecithin and 75 g 5-hydroxytryptophan, and mixed uniformly to obtain a target composition for relieving stress and enhancing immunity.

### Embodiment 8

200 g turmeric extract, 100 g sea cucumber extract, 250 g chlorella extract, 100 g cranberry extract, 100 g ginseng extract, 150 g coconut oil extract, and 200 g palm oil extract were weighed, mixed the components uniformly, then, added 75 g lecithin and 80 g 5-hydroxytryptophan, and mixed uniformly to obtain a target composition for relieving stress and enhancing immunity.

### Embodiment 9

300 g turmeric extract, 250 g sea cucumber extract, 175 g chlorella extract, 50 g cranberry extract, 300 g ginseng extract, 150 g coconut oil extract, and 100 g palm oil extract were weighed, mixed the components uniformly, then, added 100 g lecithin and 80 g 5-hydroxytryptophan, and mixed uniformly to obtain a target composition for relieving stress and enhancing immunity.

### Embodiment 10

Added pharmaceutically acceptable adjuvants into the composition for relieving stress and enhancing immunity prepared in Embodiment 1 to prepare a tablet, wherein, a content of the active ingredient was 40% by weight.

### Embodiment 11

Added pharmaceutically acceptable adjuvants into the composition for relieving stress and enhancing immunity prepared in Embodiment 2 to prepare a capsule, wherein, a content of the active ingredient was 20% by weight.

### Embodiment 12

Added pharmaceutically acceptable adjuvants into the composition for relieving stress and enhancing immunity prepared in Embodiment 4 to prepare a granule, wherein, a content of the active ingredient was 30% by weight.

### Embodiment 13

Added pharmaceutically acceptable adjuvants into the composition for relieving stress and enhancing immunity prepared in Embodiment 6 to prepare a pill, wherein, a content of the active ingredient was 25% by weight.

### Embodiment 14

Added pharmaceutically acceptable adjuvants into the composition for relieving stress and enhancing immunity prepared in Embodiment 8 to prepare a powder, wherein, a content of the active ingredient was 18% by weight.

### Embodiment 15

The composition for relieving stress and enhancing immunity prepared in Embodiment 2 was subjected to the following pharmacodynamic test:

Test animals: 80 clean-grade Kunming mice, half male and half female, having a weight of 18-22 g each.

Experimental reagent: the composition for relieving stress and enhancing immunity prepared in Embodiment 2 was prepared into a 6% suspension with distilled water for use.

Experimental method: the experimental animals were randomly divided into 4 groups, 20 in each group: the control group was intragastrically administered with normal saline 0.3 mL/each mouse per day, the experimental group was intragastrically administered with 0.3 mL suspension per day, the concentrations of the gastric perfusion suspensions of the 3 experimental groups were 3 g/(kg·bw), 5 g/(kg·bw), and 7 g/(kg·bw), respectively, twice a day for 2 weeks. Each mouse was intraperitoneally injected with 1 mL of 20% chicken red blood cell suspension. After 30 minutes, the mice were sacrificed by cervical dislocation and fixed on the wax plate. The skin of the abdominal wall was cut from the median, 2 mL of normal saline was injected through the abdominal cavity, and the rat plate was rotated for 1 min to fully mix the normal saline and peritoneal fluid, and then aspirated 1 mL of the peritoneal washing solution, which were then averaged to drip on 2 sheets of slides, placed in an enamel box with wet gauze, and incubated in a 37° C. incubator for 30 min. After incubation, the slides were removed and rinsed in normal saline to remove the unpatched cells. After drying, it was fixed in a 1:1 acetone methanol solution for 20 min and dyed using the Giemsa dye solution for 3 min, then rinsed with distilled water, dried, and counted the macrophages under a microscope. Calculated the phagocytosis rate and the phagocytosis index using the formula.

Phagocytosis rate (%)=macrophages that phagocytose chicken red blood cells/counted macrophages×100%, phagocytosis index=total number of the swallowed chicken red blood cells/counted macrophages.

The results are shown in Table 1.

TABLE 1
Effect of composition for relieving stress and enhancing
immunity prepared by Embodiment 2 on phagocytosis
of mouse peritoneal macrophages(X̅ ± s)

| | | phagocytosis | phagocytosis |
|---|---|---|---|
| groups | numbers | rate (%) | index |
| Control group | 20 | 39.3 ± 9.5 | 0.52 ± 0.23 |
| Low-dose | 20 | 48.4 ± 10.1 | 1.37 ± 0.31 |
| experimental group | | | |
| 3 g/(kg · bw) | | | |
| Medium-dose | 20 | 59.8 ± 9.5 | 1.86 ± 0.29 |
| experimental group | | | |
| 5 g/(kg · bw) | | | |
| High-dose | 20 | 68.7 ± 11.6 | 1.63 ± 0.43 |
| experimental group | | | |
| 7 g/(kg · bw) | | | |

Note:
compared with the control, P < 0.05.

The experimental results show that the low, medium and high dose groups of the suspension of the composition for relieving stress and enhancing immunity prepared in Embodiment 2 can effectively increase the phagocytosis rate and phagocytosis index of mouse macrophages, indicating that the obtained compositions for relieving stress and enhancing immunity can increase the phagocytic activity of phagocytic cells and enhance the phagocytosis of the mononuclear phagocytic system.

### Embodiment 16

Experiments with compositions for relieving stress and enhancing immunity prepared in Embodiment 1, Embodiment 3, Embodiment 4, Embodiment 5, Embodiment 6, Embodiment 7, Embodiment 8, and Embodiment 9, respectively, were carried out, methods were same as shown in Embodiment 15, the results all showed that the compositions for relieving stress and enhancing immunity of the present invention can enhance the phagocytic function of mouse peritoneal macrophages.

What is claimed is:

1. A composition for relieving stress and enhancing immunity, characterized in, the composition for relieving stress and enhancing immunity is calculated in parts by weight, 100-300 parts of a turmeric extract, 100-250 parts of a sea cucumber extract, 100-250 parts of a chlorella extract, 50-150 parts of a cranberry extract, 100-300 parts of a ginseng extract, 80-150 parts of a coconut oil extract, and 100-200 parts of a palm oil extract.

2. The composition for relieving stress and enhancing immunity according to claim 1, characterized in, the composition for relieving stress and enhancing immunity is calculated in parts by weight, further comprising 50-100 parts of lecithin and 50-80 parts of 5-hydroxytryptophan.

3. The composition for relieving stress and enhancing immunity according to claim 1, characterized in, the composition for relieving stress and enhancing immunity is calculated in parts by weight, 150-250 parts of a turmeric extract, 100-200 parts of a sea cucumber extract, 150-200 parts of a chlorella extract, 80-100 parts of a cranberry extract, 150-250 parts of a ginseng extract, 100-150 parts of a coconut oil extract, 150-200 parts of a palm oil extract, 50-100 parts of lecithin and 50-80 parts of 5-hydroxytryptophan.

4. The composition for relieving stress and enhancing immunity according to claim 2, characterized in, the composition for relieving stress and enhancing immunity is calculated in parts by weight, 150-250 parts of a turmeric extract, 100-200 parts of a sea cucumber extract, 150-200 parts of a chlorella extract, 80-100 parts of a cranberry extract, 150-250 parts of a ginseng extract, 100-150 parts of a coconut oil extract, 150-200 parts of a palm oil extract, 50-100 parts of lecithin and 50-80 parts of 5-hydroxytryptophan.

5. A method for preparing a composition for relieving stress and enhancing immunity according to claim 1, characterized in, mixing a turmeric extract, a sea cucumber extract, a chlorella extract, a cranberry extract, a ginseng extract, a coconut oil extract, and a palm oil extract in proportion according to the above ratio, then adding formula-proportioned lecithin and 5-hydroxytryptophan and mixing uniformly to obtain a target composition for relieving stress and enhancing immunity.

6. A method for preparing a composition for relieving stress and enhancing immunity according to claim 5, characterized in, the turmeric extract is prepared by pulverizing a turmeric medicinal material, extracting the turmeric medicinal material with a mixed solution of 50%-95% lower alcohol solution and n-hexane, the extraction time is 1-3 hours, the extraction temperature is 50° C.-85° C., and the extraction times are 2-3 times; collecting a concentrated extract, adding a 80%-90% lower alcohol solution having a volume of 0.2-0.4 times of the concentrated extract, stirring uniformly and allowing to stand for 12-24 hours, centrifuging and collecting crystals; washing the crystals again with a 80%-95% lower alcohol solution, and centrifuging to obtain crystals; drying the crystals at a low temperature to obtain a turmeric extract;
The sea cucumber extract is prepared by washing a sea cucumber with cold water, adding cold purified water at 4° C. to the sea cucumber and sealing, and soaking in cold storage at 4° C. for 48 hours; then homogenizing, freezing at −20° C. until completely frozen, then thawing at room temperature, centrifuging at 4° C. and collecting a supernatant and a precipitate; filtering the supernatant through a sterile membrane with a pore size of 0.25 μm, and storing a filtrate at 4° C. for use; freezing and thawing the resulting precipitate again by adding water, centrifuging and collecting a supernatant; blending the two supernatants and concentrating under reduced pressure, filtering and lyophilizing a concentrated sea cucumber extract to obtain a sea cucumber extract;
The chlorella extract is prepared by pulverizing a chlorella, dispersing uniformly with water having a weight of 10-12 times of the chlorella to obtain a dispersion; centrifuging and separating the dispersion, taking a supernatant, filtering, decolorizing and lyophilizing to obtain a chlorella extract.

7. A method for preparing a composition for relieving stress and enhancing immunity according to claim 5, characterized in, the cranberry extract is prepared by pulverizing a cranberry, extracting with an acidic methanol, extracting at 80-100° C. for 10-20 minutes, and then drying into a powder;
The ginseng extract is prepared by pulverizing a ginseng, soaking in water, vacuum distilling, stopping distillation when a volume of a distillate is half of the amount of added water, and collecting the distillate; and then continuously adding water to a liquid, vacuum distilling, stopping distillation when a volume of a distillate is half of the amount of added water, and collecting the distillate; blending the two distillates to obtain a ginseng extract.

8. A method for preparing a composition for relieving stress and enhancing immunity according to claim 5, characterized in, the coconut oil extract is prepared by repeatedly extracting a natural coconut oil by alcohol, and lyophilizing an extract to obtain a coconut oil extract;
The palm oil extract is prepared by repeatedly extracting a natural palm oil by alcohol and lyophilizing an extract to obtain a palm oil extract.

9. A formulation for relieving stress and enhancing immunity, characterized in, the composition according to claim 1 is prepared as an active ingredient together with pharmaceutically acceptable adjuvants to obtain a forming agent; wherein, a content of the active ingredient is 16 wt %-40 wt %.

10. A formulation for relieving stress and enhancing immunity, characterized in, the composition according to claim 2 is prepared as an active ingredient together with pharmaceutically acceptable adjuvants to obtain a forming agent; wherein, a content of the active ingredient is 16 wt %-40 wt %.

11. A formulation for relieving stress and enhancing immunity, characterized in, the composition according to claim 3 is prepared as an active ingredient together with pharmaceutically acceptable adjuvants to obtain a forming agent; wherein, a content of the active ingredient is 16 wt %-40 wt %.

12. A formulation for relieving stress and enhancing immunity, characterized in, the composition according to claim 4 is prepared as an active ingredient together with pharmaceutically acceptable adjuvants to obtain a forming agent; wherein, a content of the active ingredient is 16 wt %-40 wt %.

13. A formulation for relieving stress and enhancing immunity, characterized in, the composition according to claim 5 is prepared as an active ingredient together with pharmaceutically acceptable adjuvants to obtain a forming agent; wherein, a content of the active ingredient is 16 wt %-40 wt %.

14. The formulation for relieving stress and enhancing immunity according to claim 1, characterized in, a dosage form of the formulation is a tablet, a capsule, a granule, a pill or a powder.
